(19) United States
(12) Patent Application Publication
(10) Pub. No.: US 2022/0370351 A1
(43) Pub. Date: Nov. 24, 2022

(54) THREE-DIMENSIONAL FLOW PATH STRUCTURE BODY AND NANOPARTICLE PRODUCTION METHOD USING SAME

(71) Applicant: National University Corporation Hokkaido University, Sapporo-shi, Hokkaido (JP)

(72) Inventors: Masatoshi Maeki, Sapporo-Shi, Hokkaido (JP); Manabu Tokeshi, Sapporo-Shi, Hokkaido (JP); Hideyoshi Harashima, Sapporo-Shi, Hokkaido (JP); Yusuke Sato, Sapporo-Shi, Hokkaido (JP); Niko Kimura, Sapporo-Shi, Hokkaido (JP)

(21) Appl. No.: 17/765,893

(22) PCT Filed: Oct. 4, 2019
(86) PCT No.: PCT/JP2019/039347

§ 371 Date: Apr. 1, 2022

(51) Int. Cl. A61K 9/127; C12N 15/113; B01J 13/04

(52) U.S. Cl. CPC A61K 9/127; C12N 15/113; B01J 13/04; C12N 2310/14; C12N 2320/32

(57) ABSTRACT

The present invention pertains to a flow path structure body for forming self-assembling molecular particles. The flow path structure body has a base body and a flow path structure provided to the interior thereof, the flow path structure having a first introduction channel 10 and a second introduction channel 20 that are independent of one another on the upstream side of the flow path structure, and the introduction channels merging at a merging site. The flow path structure has a dilution flow path 40 that is bent three-dimensionally toward the downstream side of the merging site. The dilution flow path 40 has two or more Y structural elements 50 that protrude out in the Y direction and one or more Z structural elements 60 that protrude out in the Z direction within the dilution flow path, and at least two adjacent Y structural elements protrude out alternately in the Y direction. The present invention pertains to a method of producing self-assembling molecular particles, whereby a self-assembling molecule-containing solution and a dilution medium are supplied to the flow path structure body to form self-assembling molecular particles that have encapsulated a substance to be encapsulated. The present invention provides: a method of producing self-assembling molecular nanoparticles that enables precise control of the particle size of self-assembling molecular nanoparticles that have encapsulated an anionic molecule or the like at a high encapsulation rate; and a flow path structure body used for production.

### TECHNICAL FIELD

The present invention relates to a three-dimensional flow path structure body and to a nanoparticle production method that uses same.

### BACKGROUND ART

The present invention relates to a three-dimensional flow path structure body and to a nanoparticle production method that uses same.

The practical application of nanoparticles that contain self-assembling molecules as a particle constituent component, e.g., lipid nanoparticles and polymer micelles, is proceeding most prominently in the area of use as nanocarriers for drug delivery systems (DDS), and clinical applications are already underway. It has been quite recently found that the drug delivery efficiency to cancer tissue varies depending on the nanocarrier particle diameter. Particle diameter control methods that use microfluidic devices are therefore being developed in order to precisely control the nanoparticle particle diameter [NPL 2 to 4].

In addition, the present inventors have developed a microfluidic device that is easy to fabricate and process and that exhibits a high particle diameter control performance, and have also developed a nanoparticle production method that uses this microfluidic device [NPL 1 and PTL 1]. The nanoparticle production methods described in NPL 1 and PTL 1 enable a precise particle diameter control by carrying out the very rapid dilution, for example, of a lipid/alcohol solution that is a starting material for the production of lipid nanoparticles with a buffer in the microfluidic flow path.

### CITATION LIST

### Patent Literature

[PTL 1] WO 2018/190423
[PTL 2] Japanese Patent Application Laid-open No. 2009-505957
[PTL 3] Japanese Translation of PCT Application No. 2013-510096

### Non Patent Literature

[NPL 1] “Development of the iLiNP Device: Fine Tuning the Lipid Nanoparticle Size within 10 nm for Drug Delivery”, N. Kimura, M. Maeki, Y. Sato, T. Note, A. Ishida, H. Tani, H. Harashima, and M. Tokeshi, ACS Omega, 3, 5044, (2018).
[NPL 2] “Understanding the Formation Mechanism of Lipid Nanoparticles in Microfluidic Devices with Chaotic Micromixers”, M. Maeki, Y. Fujishima, Y. Sato, T. Yasui, N. Kaji, A. Ishida, H. Tani, Y. Baba, H. Harashima, and M. Tokeshi, PLOS ONE, 12, e0187962, (2017).
[NPL 3] “Bottom-Up Design and Synthesis of Limit Size Lipid Nanoparticle Systems with Aqueous and Triglyceride Cores Using Millisecond Microfluidic Mixing”, I. V. Zhigaltsev, N. Belliveau, I. Hafez, A. K. K. Leung, C. Hansen, and P. R. Cullis, Langmuir, 38, 3633, (2012).
[NPL 4] “Rapid Discovery of Protein siRNA-Containing Lipid Nanoparticles Enabled by Controlled Microfluidic Formation”, D. Chen, K. T. Love, Y. Chen, A. A. Eltoukhy, C. Kastrup, G. Sahay, A. Jeon, Y. Dong, K. A. Whitehead, and D. G. Anderson, Journal of the American Chemical Society, 134, 6948, (2012).

### SUMMARY OF INVENTION

### Technical Problem

Nucleic acids are an example of bioactive substances that are encapsulated into self-assembling molecular nanoparticles. PTL 1 to PTL 3 and NPL 1 disclose the encapsulation of nucleic acids into cationic lipid nanoparticles. Since nucleic acids are anionic, they are relatively easy to be encapsulated in cationic lipid nanoparticles, which have the opposite charge. However, cationic lipids can be adsorbed to proteins in the blood and also pose the problem of cell toxicity. Neutral lipids and anionic lipids exist in addition to cationic lipids, but neutral lipids lack charge and lack the ability to attract nucleic acids. Anionic lipids have the same charge as nucleic acids, which are anionic, and, due to electrostatic repulsion, encapsulation in lipid nanoparticles has been much more difficult than with anionic lipids. This feature also applies to self-assembling molecules other than lipids. While the example of nucleic acids has been used here, the same feature also applies when the encapsulation of other anionic molecules (small molecules, middle molecules, macromolecules) is sought.

An object of the present invention is to provide a method for producing self-assembled molecular nanoparticles, wherein this method can produce—even in the case of use of neutral self-assembling molecules and anionic self-assembling molecules—self-assembled molecular nanoparticles in which anionic molecules (the description is centered on nucleic acids in the following, but there is no limitation to this) are encapsulated at high encapsulation efficiency, and wherein this method is also capable of a precise control of the particle diameter of the self-assembled molecular nanoparticles. An object of the present invention is also to provide a flow path structure body for use in this production.

### Solution to Problem

The present invention is as follows.

[1]

A flow path structure body for the formation of a particle (referred to below as a self-assembled molecular particle) that contains self-assembling molecules as a particle constituent component, wherein

the flow path structure body has a base body and a flow path structure disposed in the interior thereof;

the flow path structure has, on its upstream side, at least two introduction channels that are independent from each other and are a first introduction channel that introduces a first liquid and a second introduction channel that introduces a second liquid, wherein these introduction channels merge at a merge site;

the flow path structure has at least one three-dimensionally bent dilution flow path toward the downstream side of the merge site; and

designating the X direction to be the axial direction of the dilution flow path upstream from the three-dimensionally bent dilution flow path, or the direction of the extension of this axial direction, designating the Y direction to be the width direction of the dilution flow path that perpendicularly intersects the X direction, and designating the Z direction to be the depth direction of the dilution flow path that perpendicularly intersects each of the X direction and Y direction, two or more Y structural elements that protrude in the Y direction and one or more Z structural element that protrudes in the Z direction are each independently present in at least a portion of the dilution flow path and at least two adjacent Y structural elements alternately protrude in the Y direction.

[2]

The flow path structure body according to [1], wherein a Y structural element has a protrusion length that is in the range of ±10 to 90% of the flow path width (here, + is the protrusion length from one inner surface of the flow path, and − is the protrusion length from the flow path inner surface that is disposed opposite to said one inner surface of the flow path), has a length in the X direction that is in the range of 10% to 1,000% of the flow path width, and has an interval for the opposing sides of two adjacent Y structural elements in the range of 10% to 1,000% of the flow path width.

[3]

The flow path structure body according to [1] or [2], wherein a Z structural element has a protrusion length that is in the range of ±10 to 90% of the flow path height (here, + is the protrusion length from one inner surface of the flow path, and − is the protrusion length from the flow path inner surface that is disposed opposite to said one inner surface of the flow path), has a length in the X direction that is in the range of 10% to 1,000% of the flow path width, and, when there are two or more Z structural elements, has an interval for the opposing sides of two adjacent Z structural elements that is at least 10% of the flow path width.

[4]

The flow path structure body according to any of [1] to [3], wherein the length x, width y, and height z of the dilution flow path from the merge site of the introduction channels to the first Y structural element and/or the first Z structural element are x:y in the range of 1 to 100:1 and z:y in the range of 0.1 to 5:1.

[5]

The flow path structure body according to any of [1] to [4], wherein the first structural element from the merge site of the introduction channels is a Y structural element, a Z structural element, or a Y structural element plus a Z structural element.

[6]

The flow path structure body according to any of [1] to [5], wherein the first structural element from the merge site of the introduction channels is a Y structural element (in the following, this is referred to as the first Y structural element and the downstream Y structural elements are referred to in sequence as the m^{th} Y structural elements where m is an integer greater than or equal to 2) plus a Z structural element (in the following, this is referred to as the first Z structural element and the downstream Z structural elements are referred to in sequence as the n^{th} Y structural elements where n is an integer greater than or equal to 2);

the length in the X direction of the first Y structural element is the same as the length in the X direction of the first Z structural element; and

the interval between the first Z structural element and a second Z structural element is equal to the interval between the first Y structural element and a second Y structural element.

[7]

The flow path structure body according to [6], wherein, for downstream from the second Z structural element and second Y structural element, the disposition positions of Z structural elements and the disposition positions of Y structural elements have the same relationship as the positional relationship described in [6].

[8]

The flow path structure body according to any of [1] to [5], wherein

the first structural element from the merge site for the introduction channels is a Y structural element;

the upstream side surface of the first Z structural element, which is the first Z structural element from the merge site for the introduction channels, is at the same position as the upstream side surface of the second Y structural element;

the length in the X direction of the first Z structural element is the same as the length in the X direction of the second Y structural element; and

the interval between the first Z structural element and the second Z structural element is equal to the interval between the second Y structural element and a fourth Y structural element.

[9]

The flow path structure body according to [8], wherein, for downstream from the second Z structural element and the fourth Y structural element, the disposition positions of Z structural elements and the disposition positions of Y structural elements have the same relationship as the positional relationship described in [8].

[10]

The flow path structure body according to any of [1] to [5], wherein

the first structural element from the merge site of the introduction channels is a Y structural element plus a Z structural element;

the upstream side surface of the first Z structural element, which is the first Z structural element from the merge site for the introduction channels, is at the same position as the upstream side surface of the first Y structural element;

the length in the X direction of the first Z structural element is the same as the length in the X direction of the first Y structural element; and

the interval between the first Z structural element and the second Z structural element is equal to the interval between the first Y structural element and a third Y structural element.

[11]

The flow path structure body according to [10], wherein, for downstream from the second Z structural element and a fourth Y structural element, the disposition positions of Z structural elements and the disposition positions of Y structural elements have the same relationship as the positional relationship described in [10].

[12]

The flow path structure body according to any of [1] to [11], wherein the number of Y structural elements is in the range of 3 to 100 and the number of Z structural elements is in the range of 2 to 100.

[13]

The flow path structure body according to any of [1] to [12], wherein, with respect to the dilution flow path from the merge site of the introduction channels to the first Y structural element and/or the first Z structural element, the length x is in the range from 20 to 1,000 μm, the width y is in the range from 20 to 1,000 μm, and the height z is in the range from 20 to 1,000 μm.

[14]

The flow path structure body according to any of [1] to [13], wherein the first introduction channel and the second introduction channel intersect with the merge site at an angle, for each independently, of 10° to 90° versus the flow path direction (X direction).

[15]

The flow path structure body according to any of [1] to [14], wherein the first introduction channel includes a plurality of flow paths and/or the second introduction channel includes a plurality of flow paths.

[16]

A method for producing a self-assembling molecular particle, including supplying a self-assembling molecule-containing solution and a dilution medium to a flow path structure body, and forming a self-assembling molecular particle that encapsulates a substance to be encapsulated, wherein

at least one of the self-assembling molecule-containing solution and the dilution medium contains a substance to be encapsulated;

the flow path structure body is a flow path structure body according to any of [1] to [15]; and

the self-assembling molecular particle is obtained by introducing the self-assembling molecule-containing solution from one of the first introduction channel and the second introduction channel of this flow path structure body, introducing the dilution medium from the other introduction channel, and diluting the self-assembling molecule-containing solution with the dilution medium in the dilution flow path.

[17]

The production method according to [16], wherein the self-assembling molecule-containing solution and the dilution medium are introduced into the flow path structure body at a total flow rate of 1 μl/minute to 100 ml/minute.

[18]

The production method according to [16] or [17], wherein, with regard to the total flow rate of the self-assembling molecule-containing solution and dilution medium, the total flow rate of the self-assembling molecule-containing solution and dilution medium is established—and/or the interval from the first introduction channel/second introduction channel merge site of the flow path structure body to the upstream side end of the first structural element is established—such that the self-assembling molecule-containing solution and dilution medium traverse the interval from the first introduction channel/second introduction channel merge site to the upstream side end of the first structural element in a time that is less than or equal to 0.1 second.

[19]

The production method according to any of [16] to [18], wherein the self-assembling molecule-containing solution is at least one selection from the group consisting of neutral lipid-containing solutions, anionic lipid-containing solutions, cationic lipid-containing solutions, and polymer-containing solutions.

[20]

The production method according to any of [16] to [19], wherein the substance to be encapsulated is a nucleic acid.

[21]

The production method according to any of [16] to [20], wherein the self-assembling molecular particle having the encapsulated substance to be encapsulated has a number-average particle diameter in the range of 20 to 200 nm.

### Advantageous Effects of Invention

The present invention can thus provide a method for producing self-assembled molecular nanoparticles, wherein this method can produce—even with anionic molecules—self-assembled molecular nanoparticles that have carried out encapsulation at high encapsulation ratios, and wherein this method is also capable of a precise control of the particle diameter of the self-assembled molecular nanoparticles. The present invention can also provide a flow path structure body for use in this production.

### BRIEF DESCRIPTION OF DRAWINGS

FIG. 1-1 gives schematic diagrams and enlarged partial diagrams of an example of the flow path structure body according to the present invention having a three-dimensionally bent dilution flow path (also referred to hereinbelow as a 3D flow path structure body).

FIG. 1-2 describes an example of the 3D flow path structure body according to the present invention with division into the x-y cross section and the x-z cross section.

FIG. 1-3 shows a flow path structure 1 of the 3D flow path structure body according to the present invention, as a perspective view from an angle different from that in FIG. 1-1 and in a partial transparent perspective view.

FIG. 1-4 shows a schematic perspective explanatory diagram of embodiments A to C of the 3D flow path structure body according to the present invention.

FIG. 2 gives an explanatory diagram of an example and a comparative example (schematic diagram of an example of a flow path structure body having a two-dimensionally bent dilution flow path (also referred to hereinbelow as the 2D flow path structure body)).

FIG. 3 gives comparative results for the siRNA encapsulation ratio for a cationic lipid, neutral lipid, and anionic lipid, for Example 1 (3D flow path structure body=basic structure) and a comparative example.

FIG. 4 gives comparative results for the number-average particle diameter and siRNA encapsulation ratio for an anionic lipid, for Example 1 (3D flow path structure body=basic structure) and a comparative example.

FIG. 5 gives comparative results for the number-average particle diameter and siRNA encapsulation ratio for a neutral lipid, for Example 1 (3D flow path structure body=basic structure) and a comparative example.

FIG. 6 is a schematic explanatory diagram of the structure of the bent flow path of the 3D flow path structure body used in Example 2.

FIG. 7 reports, in a comparative regime, the results for the number-average particle diameter and Z-average particle diameter for the anionic lipid in Example 2.

FIG. 8 reports, in a comparative regime, the particle size distribution for the number-average particle diameter for the anionic lipid in Example 2.

FIG. 9 reports, in a comparative regime, the particle size distribution for the Z-average particle diameter for the anionic lipid in Example 2.

FIG. 10 reports, in a comparative regime, the siRNA encapsulation ratio for the anionic lipid in Example 2.

FIG. 11 reports, in a comparative regime, the results for the number-average particle diameter and Z-average particle diameter for the neutral lipid in Example 2.

FIG. 12 reports, in a comparative regime, the siRNA encapsulation ratio for the neutral lipid in Example 2.

FIG. 13 is a schematic explanatory diagram of the structure of the bent flow path of the 3D flow path structure body used in Example 3.

FIG. 14 reports, in a comparative regime, the results for the number-average particle diameter and Z-average particle diameter for the anionic lipid in Example 3.

FIG. 15 reports, in a comparative regime, the particle size distribution for the number-average particle diameter for the anionic lipid in Example 3.

FIG. 16 reports, in a comparative regime, the particle size distribution for the Z-average particle diameter for the anionic lipid in Example 3.

FIG. 17 reports, in a comparative regime, the siRNA encapsulation ratio for the anionic lipid in Example 3.

FIG. 18 reports, in a comparative regime, the results for the number-average particle diameter and Z-average particle diameter for the neutral lipid in Example 3.

FIG. 19 reports, in a comparative regime, the siRNA encapsulation ratio for the neutral lipid in Example 3.

FIG. 20 is a schematic explanatory diagram of the structure of the bent flow path of the 3D flow path structure body used in Example 4.

FIG. 21 reports, in a comparative regime, the results for the number-average particle diameter and Z-average particle diameter for the anionic lipid in Example 4.

FIG. 22 reports, in a comparative regime, the particle size distribution for the number-average particle diameter for the anionic lipid in Example 4.

FIG. 23 reports, in a comparative regime, the particle size distribution for the Z-average particle diameter for the anionic lipid in Example 4.

FIG. 24 reports, in a comparative regime, the siRNA encapsulation ratio for the anionic lipid in Example 4.

FIG. 25 reports, in a comparative regime, the results for the number-average particle diameter and Z-average particle diameter for the neutral lipid in Example 4.

FIG. 26 reports, in a comparative regime, the siRNA encapsulation ratio for the neutral lipid in Example 4.

FIG. 27 reports the results of the evaluations in Example 5 for an in vivo experiment.

FIG. 28 reports the results of the knock down activity in Example 5.

FIG. 29 reports the results of a simulation for a total flow rate of 500 μl/minute in Reference Example 1.

FIG. 30 reports the results of a simulation for a total flow rate of 500 μl/minute in Reference Example 1.

FIG. 31 reports the results of a simulation for a total flow rate of 50 μl/minute in Reference Example 1.

FIG. 32 reports the results of a simulation for a total flow rate of 50 μl/minute in Reference Example 1.

### DESCRIPTION OF EMBODIMENTS

(Flow Path Structure Body)

The flow path structure body according to the present invention is a flow path structure body for forming self-assembling molecular particles. This flow path structure body has a base body and a flow path structure disposed in this base body. On its upstream side, the flow path structure has at least two introduction channels that are independent from each other and are a first introduction channel that introduces a first liquid and a second introduction channel that introduces a second liquid. The at least two introduction channels merge at a merge site, and the flow path structure has at least one three-dimensionally bent dilution flow path toward the downstream side of the merge site. The three-dimensionally bent dilution flow path has, within at least a portion of this dilution flow path, two or more Y structural elements that protrude in the Y direction of the flow path interior and one or more Z structural element that protrudes in the Z direction of the flow path interior—these structural elements being present each independently—and at least two adjacent Y structural elements alternately protrude in the Y direction. The Y structural element and Z structural element protrude toward the flow path interior of the dilution flow path. Here, the X direction is the axial direction of the dilution flow path upstream from the three-dimensionally bent portion, or the direction of the extension of this axial direction; the Y direction is the width direction of the dilution flow path that perpendicularly intersects with the X direction; and the Z direction is the depth direction of the dilution flow path that perpendicularly intersects with each of the X direction and Y direction.

The self-assembling molecular particles that are formed by the flow path structure body according to the present invention are described below.

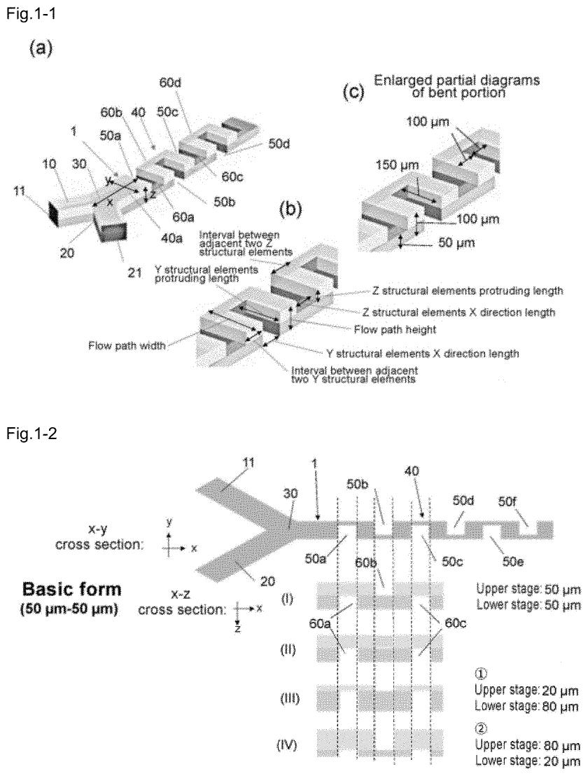

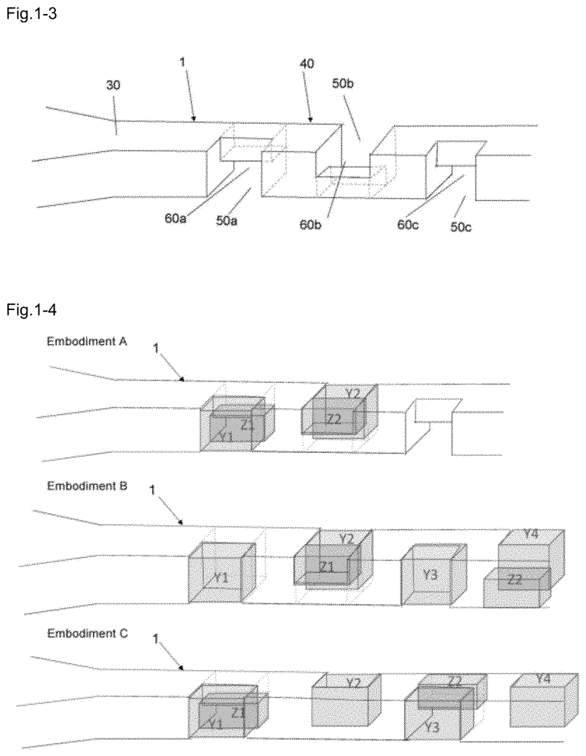

The flow path structure body according to the present invention is described in detail in the following based on FIG. 1.

A flow path structure 1 of the flow path structure body according to the present invention is shown in FIG. 1-1(a). While not shown in the figure, the flow path structure body includes a base body and a flow path structure disposed in the interior of this base body. The base body includes a hard or soft material, e.g., resin and so forth, and at least a portion of the flow path structure 1 is formed as a tubular structure in the interior of the base body. The shape of the horizontal cross section of the tubular structure is not particularly limited and may be, for example, rectangular (for example, square, rectangular, rhombic), polygonal (for example, triangular, pentagonal, hexagonal, octagonal), or circular (for example, true circle, ellipse, oval) or any combination of rectangular, polygonal, and circular. In addition, the shape of the horizontal cross section may be the same regardless of the region in the flow path structure, or the shape of the horizontal cross section may vary depending on the region in the flow path structure. FIGS. 1-1, 1-2, 1-3, 2, 6, 13, 20, and 29 to 32 describe only the flow path structure formed in the interior of the base body. In addition, in the flow path structures shown in the individual figures, two types of shading are present for the sake of convenience depending on the region, but this does not represent differences in structure or the presence of partition walls, and the interior of the flow path structure is a single continuous space without, for example, partition walls.

On its upstream side, the flow path structure 1 of the flow path structure body according to the present invention has at least two introduction channels that are independent from each other and are a first introduction channel 10 that introduces a first liquid and a second introduction channel 20 that introduces a second liquid. The first introduction channel 10 and the second introduction channel 20 merge at a merge site 30. At least one three-dimensionally bent dilution flow path 40 is present toward the downstream side of the merge site 30. The three-dimensionally bent dilution flow path 40 has, in at least a portion of this dilution flow path, two or more Y structural elements 50a, 50b . . . that protrude in the Y direction and one or more Z structural element 60a, 60b . . . that protrudes in the Z direction, which are each independently present. Only the Y structural elements and Z structural elements in a portion of the upstream portion of the dilution flow path 40 are shown in FIGS. 1-1 to 1-3. The Y structural elements and Z structural elements function as obstacles or baffle plates that alter the direction of flow of the fluid flowing through the interior of the dilution flow path. At least two adjacent Y structural elements have structures that protrude alternately in the Y direction. This serves to promote the dilution, by the dilution medium, of the self-assembling molecules in the self-assembling molecule-containing solution, and to promote the formation of the particles thereby produced. Preferably three or more and more preferably all of the adjacent Y structural elements have structures that protrude alternately in the Y direction. In the flow path structure of the flow path structure body according to the present invention, the dilution, by the dilution medium, of the self-assembling molecules in the self-assembling molecule-containing solution and the formation of the particles thereby produced are more substantially promoted, as compared to the presence of only Y structural elements, by the disposition of at least one Z structural element in addition to the Y structural elements.

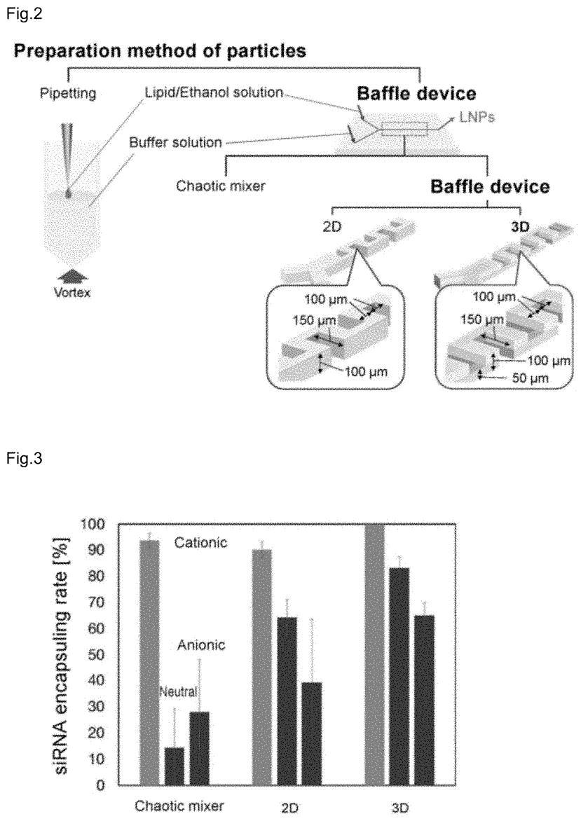

A plurality of Y structural elements 50a, 50b, 50c, 50d and Z structural elements 60a, 60b, 60c, 60d are shown in the flow path structure 1 of the flow path structure body according to the present invention in FIG. 1-1(a). However, the Z structural elements 60b and 60d are behind the flow path and are not visible in the figure. The flow path structure 1 of the flow path structure body according to the present invention is described in FIG. 1-2 with partitioning into the x-y cross section and the x-z cross section. Four embodiments are shown: (I) for the x-z cross section is an embodiment in which the thicknesses of the upper stage and lower stage are both 50 μm (also referred to as the basic shape in the examples); (II) is an embodiment in which the thickness of the upper stage and the thickness of the lower stage are both 50 μm, but the Z structural elements shown in the x-z cross section are 60a and 60c and 60b is absent; (Ill) is an embodiment in which the thicknesses of the upper stage and lower stage are 20 μm and 80 μm; and (IV) is an embodiment in which the thicknesses of the upper stage and lower stage are 80 μm and 20 μm. FIG. 1-3 shows a flow path structure 1 of the flow path structure body according to the present invention, as a perspective diagram from an angle different from that in FIG. 1-1 and in a partial transparent perspective view. FIG. 1-3 elucidates the relationship between the Y structural element 50a and the Z structural element 60a, the relationship between the Y structural element 50b and the Z structural element 60b, and the relationship between the Y structural element 50c and the Z structural element 60c. In the embodiments shown in FIGS. 1-1 to 1-3, two or more Y structural elements and two or more Z structural elements are each disposed in alternation, but this should not be understood as a limitation on the present invention. For the Y structural elements, at least two adjacent Y structural elements should have structures that alternately protrude in the Y direction, and at least one Z structural element should be provided.

The first introduction channel 10 has, at the most upstream position, an introduction port 11 for the introduction of a first liquid, and the second introduction channel 20 has, at the most upstream position, an introduction port 21 for the introduction of a second liquid. The dilution flow path 40 has, at the most downstream position, a discharge port (not shown) for discharging the fluid containing self-assembling molecular particles that have been formed in the flow path structure 1.

As shown in FIG. 1-1(b), the Y structural element has a protrusion length, for example, that can be in the range of ±10 to 90% of the flow path width. Here, + is the protrusion length from one inner surface of the flow path, and − is the protrusion length from the flow path inner surface that is disposed opposite to said one inner surface of the flow path. The protrusion length of the Y structural element is preferably in the range of ±20 to 80% of the flow path width, more preferably in the range of ±30 to 70% of the flow path width, and even more preferably in the range of ±40 to 60% of the flow path width. The length in the X direction of the Y structural element can be, for example, in the range of 10% to 1,000% of the flow path width, preferably in the range of 20% to 500% of the flow path width, more preferably in the range of 30% to 300% of the flow path width, even more preferably in the range of 40% to 200% of the flow path width, and still more preferably in the range of 50% to 150% of the flow path width. The interval for the opposing sides of two adjacent Y structural elements can be, for example, in the range of 10% to 1,000% of the flow path width, preferably in the range of 20% to 500% of the flow path width, more preferably in the range of 30% to 300% of the flow path width, still more preferably in the range of 40% to 200% of the flow path width, and even more preferably in the range of 50% to 150% of the flow path width.

As shown in FIG. 1-1(b), the Z structural element has a protrusion length that, for example, can be in the range of ±10 to 90% of the flow path height. Here, + is the protrusion length from one inner surface of the flow path, and − is the protrusion length from the flow path inner surface that is disposed opposite to said one inner surface of the flow path. The protrusion length of the Z structural element is preferably in the range of ±20 to 80% of the flow path height, more preferably in the range of ±30 to 70% of the flow path height, and still more preferably in the range of ±40 to 60% of the flow path height. The embodiment (Ill), in which the thickness of the upper stage is 20 μm and the thickness of the lower stage is 80 μm, and the embodiment (IV), in which the thickness of the upper stage is 80 μm and the thickness of the lower stage is 20 μm, are provided as examples in the x-z cross section in FIG. 1-2. The length in the X direction of the Z structural element can be, for example, in the range of 10% to 1,000% of the flow path width, preferably in the range of 20% to 500% of the flow path width, more preferably in the range of 30% to 300% of the flow path width, even more preferably in the range of 40% to 200% of the flow path width, and still more preferably in the range of 50% to 150% of the flow path width. The interval for the opposing sides of two adjacent Z structural elements can be, for example, in the range of 10% to 1,000% of the flow path width, preferably in the range of 100% to 500% of the flow path width, more preferably in the range of 150% to 450% of the flow path width, still more preferably in the range of 200% to 400% of the flow path width, and even more preferably in the range of 250% to 350% of the flow path width.

The length x, width y, and height z of the dilution flow path 40a from the merge site of the introduction channels to the first Y structural element 50a and/or the first Z structural element 60a (refer to FIG. 1-1(a)) can be, for example, x:y in the range of 1 to 100:1 and z:y in the range of 0.1 to 5:1. x:y is preferably in the range of 1 to 50:1, more preferably in the range of 1 to 20:1, and still more preferably in the range of 1 to 10:1. z:y is preferably in the range of 0.2 to 4:1, more preferably in the range of 0.3 to 3:1, and still more preferably in the range of 0.5 to 2:1.

The first structural element from the merge site 30 of the introduction channels can be a Y structural element, a Z structural element, or a Y structural element plus a Z structural element, and a Y structural element plus a Z structural element is preferred.

In a preferred embodiment (embodiment A),

the first structural element from the merge site 30 of the introduction channels is a Y structural element plus a Z structural element, and

referring to the initial Y structural element as the first Y structural element, referring to the downstream Y structural elements in sequence as the m^{th} Y structural elements (m is an integer greater than or equal to 2), referring to the initial Z structural element as the first Z structural element, and referring to the downstream Z structural elements in sequence as the n^{th} Y structural elements (n is an integer greater than or equal to 2),

the length in the X direction of the first Y structural element is the same as the length in the X direction of the first Z structural element, and

the interval between the first Z structural element and a second Z structural element is equal to the interval between the first Y structural element and a second Y structural element. In this embodiment, the adjacent Y structural elements of a pair are in an opposing position, and the height of the Y structural elements and the height of the Z structural elements are freely selected.

Moreover, this embodiment is an example of an embodiment in which, for downstream from the second Z structural element and the second Y structural element, the disposition positions of the Z structural elements (for example, the second Z structural element and a third Z structural element) and the disposition positions of the Y structural elements (for example, the second Y structural element and a third Y structural element) have the same relationship as the interval between the first Z structural element and the second Z structural element and the interval between the first Y structural element and the second Y structural element. This embodiment corresponds to the embodiment shown in FIGS. 1-1 to 1-3. That is, the first Y structural element, second Y structural element, third Y structural element, and fourth Y structural element respectively correspond to the 50a, 50b, 50c, and 50d in the figures, while the first Z structural element, second Z structural element, third Z structural element, and fourth Z structural element respectively correspond to the 60a, 60b, 60c, and 60d in the figures.

In another embodiment (embodiment B), the first structural element from the merge site 30 for the introduction channels is a Y structural element, the upstream side surface of the first Z structural element, which is the first Z structural element from the merge site 30 for the introduction channels, is at the same position as the upstream side surface of a second Y structural element,

the length in the X direction of the first Z structural element is the same as the length in the X direction of the second Y structural element, and

the interval between the first Z structural element and a second Z structural element is equal to the interval between the second Y structural element and a fourth Y structural element. In this embodiment, the adjacent Y structural elements of a pair are in an opposing position, and the height of the Y structural elements and the height of the Z structural elements are freely selected.

In addition, this embodiment is an example of an embodiment in which, for downstream from the second Z structural element and the fourth Y structural element, the disposition positions of the Z structural elements and the disposition positions of the Y structural elements have the same relationship as described above. This embodiment is an embodiment in which one Z structural element is provided per two Y structural elements.

In another embodiment (embodiment C),

the first structural element from the merge site 30 of the introduction channels is a Y structural element plus a Z structural element,

the length in the X direction of the first Z structural element is the same as the length in the X direction of the first Y structural element, and

the interval between the first Z structural element and the second Z structural element is equal to the interval between the first Y structural element and a third Y structural element. In this embodiment, the adjacent Y structural elements of a pair are in an opposing position, and the height of the Y structural elements and the height of the Z structural elements are freely selected.

In addition, this embodiment is an example of an embodiment in which, for downstream from the second Z structural element and third Y structural element, the disposition position of the Z structural elements and the disposition position of the Y structural elements have the same relationship as described above. This embodiment is an embodiment in which the first structural element from the merge site 30 of the introduction channels is a Y structural element plus a Z structural element and in which one Z structural element is provided per two Y structural elements. In FIGS. 1-2, 50a, 50b, and 50c, corresponding to the first Y structural element, second Y structural element, and third Y structural element, are provided for the Y structural element, while, for the Z structural elements given in (II) for the x-z cross section, 60a and 60c, corresponding to the first Z structural element and second Z structural element, are present and 60b is not present.

Schematic explanatory diagrams of these embodiments A, B, and C are given in FIG. 1-4. The Y1, Y2, Y3, and Y4 in the figures denote the first Y structural element, second Y structural element, third Y structural element, and fourth Y structural element, while Z1 and Z2 indicate the first Z structural element and the second Z structural element.

There are no particular limitations other than that the number of Y structural elements is two or more and the number of Z structural elements is one or more; however, the number of Y structural elements is preferably three or more and the number of Z structural elements is preferably two or more. More specifically, the number of Y structural elements, for example, can be in the range from 3 to 100, preferably in the range from 5 to 50, more preferably in the range of 5 to 20, and still more preferably in the range from 5 to 10. The number of Z structural elements, for example, can be in the range from 2 to 100, preferably in the range from 5 to 50, more preferably in the range from 5 to 20, and still more preferably in the range from 5 to 10. In a particularly preferred case, the number of Y structural elements is in the range from 5 to 10 and the number of Z structural elements is in the range from 5 to 10.

The length x of the dilution flow path 40a from the introduction channel merge site 30 to the first Y structural element and/or the first Z structural element, for example, can be in the range from 20 to 1,000 μm and is preferably in the range from 50 to 1,000 μm and more preferably in the range from 100 to 500 μm. The width y of the dilution flow path from the introduction channel merge site 30 to the first Y structural element and/or the first Z structural element, for example, can be in the range from 20 to 1,000 μm and is preferably in the range from 50 to 500 μm, more preferably in the range from 70 to 400 μm, and still more preferably in the range from 80 to 300 μm. The height z of the dilution flow path from the introduction channel merge site 30 to the first Y structural element and/or the first Z structural element, for example, can be in the range from 20 to 1,000 μm and is preferably in the range from 50 to 500 μm, more preferably in the range from 70 to 400 μm, and still more preferably in the range from 80 to 300 μm.

The first introduction channel 10 and the second introduction channel 20 can intersect with the merge site 30 at an angle, for each independently, of 10° to 90° versus a line drawn to the upstream side in the flow path direction (X direction). This intersection angle is preferably in the range from 20° to 80°, more preferably in the range from 30° to 70°, and still more preferably in the range from 40° to 60°.

The first introduction channel 10 can be composed of a plurality of flow paths, i.e., two or more, and/or the second introduction channel 20 can be composed of a plurality of flow paths. When either introduction channel is composed of a plurality of flow paths, the plurality of flow paths is preferably 2. That is, the first introduction channel 10 and the second introduction channel 20 may also be, each independently from the other, an introduction channel formed by the merging at the upstream thereof of two or more flow paths. Alternatively, in addition to the first introduction channel 10 and the second introduction channel 20, one or more supplementary introduction channels, for example, a third introduction channel or a third introduction channel and a fourth introduction channel, may also be provided in a configuration in which merging occurs with the first introduction channel 10 and second introduction channel 20 at the merge site 30.

Either or both of the first introduction channel and the second introduction channel can be connected to a pretreatment flow path disposed on the upstream side of the introduction channel. This pretreatment flow path can be, for example, a flow path for the preparation of the self-assembling molecule-containing solution or a flow path for the preparation of the dilution medium. The pretreatment flow path can be, for example, a flow path structure body having the same structure as the flow path structure body according to the present invention. However, this should not be understood as a limitation, and a suitable selection can also be made from the heretofore known flow path structure bodies.

The discharge port can be connected to a posttreatment flow path disposed on the downstream side of the discharge port. The posttreatment flow path can be, for example, a flow path for a treatment that stabilizes the self-assembling molecular particles that have been formed. The posttreatment flow path can be, for example, a flow path structure body that has the same structure as the flow path structure body according to the present invention. However, this should not be understood as a limitation, and a suitable selection can also be made from the heretofore known flow path structure bodies.

(Method for Producing Self-Assembling Molecular Particles)

The present invention includes a method for producing self-assembling molecular particles, the method including supplying a self-assembling molecule-containing solution and a dilution medium to a flow path structure body, and forming self-assembling molecular particles that encapsulate a substance to be encapsulated. The substance to be encapsulated is contained in at least one of the self-assembling molecule-containing solution and the dilution medium. The flow path structure body in this method is the flow path structure body according to the present invention as described in the preceding, and self-assembling molecular particles are obtained by inflowing the self-assembling molecule-containing solution from one of the first introduction channel 10 and the second introduction channel 20 of this flow path structure body, introducing the dilution medium from the other introduction channel, and diluting the self-assembling molecule-containing solution with the dilution medium in the dilution flow path.

The self-assembling molecular particles formed using the flow path structure body according to the present invention and the production method according to the present invention are particles including self-assembling molecular particles as a particle constituent component. The particles including self-assembling molecules as a particle constituent component are particles obtained by the association of self-assembling molecules with each other and the formation of particles, and the substance to be encapsulated that is also present in the system during particle formation can be taken into the particle interior. The constituent components of the particles formed under conditions in which the substance to be encapsulated is also present, are at least the self-assembling molecules and the substance to be encapsulated.

The self-assembling molecule-containing solution and the dilution medium can be inflowed into the flow path structure body, for example, so as to provide a total flow rate of 1 μl/minute to 100 ml/minute. However, this should not be understood as a limitation on the total flow rate, and a suitable determination can be made considering the structure and dimensions of the flow path structure body, the type of self-assembling molecule-containing solution and dilution medium, the particle diameter desired for the self-assembling molecular particle, the encapsulation efficiency for the substance to be encapsulated, and so forth.

With regard to the total flow rate of the self-assembling molecule-containing solution and dilution medium, from the standpoint of obtaining a desired particle diameter for the self-assembling molecular particles and/or obtaining efficient encapsulation of the substance to be encapsulated, preferably the total flow rate of the self-assembling molecule-containing solution and dilution medium is established—and/or the interval from the first introduction channel/second introduction channel merge site 30 of the flow path structure body to the upstream side end of the first structural element is established—such that the self-assembling molecule-containing solution and dilution medium traverse the interval from the first introduction channel/second introduction channel merge site 30 to the upstream side end of the first structural element in a time that is less than or equal to 0.1 second.

The self-assembling molecule-containing solution can be, for example, any solution selected from the group consisting of neutral lipid-containing solutions, anionic lipid-containing solutions, cationic lipid-containing solutions, and polymer-containing solutions, but this should not be understood as a limitation to these. The self-assembling molecules in the present invention should be molecules that have a self-assembling function and, as described above, that can associate with each other and form particles. While not intended as a particular limitation, the following, for example, can be used as lipids, which are an example of self-assembling molecules: naturally-derived lipids and non-naturally-derived lipids, e.g., soy lecithin, hydrogenated soy lecithin, egg yolk lecithin, phosphatidylcholines (for example, egg-derived egg PC), phosphatidylserines, phosphatidylethanolamines, phosphatidylinositols, phosphosphingomyelins, phosphatidic acids, long-chain alkyl phosphate salts, gangliosides, glycolipids, phosphatidylglycerols, sphingolipids, sterols, and so forth; as well as non-natural-origin cationic lipids that are well suited as constituent components for nucleic acid-delivery liposomes, e.g., N,N-dioleyl-N,N-dimethylammonium chloride (DODAC), N-(2,3-dioleyloxy)propyl-N,N,N-trimethylammonium chloride (DOTMA), N,N-distearyl-N,N-dimethylammonium bromide (DDAB), N-(2,3-dioleoyloxy)propyl-N,N,N-trimethylammonium chloride (DOTAP), 3-(N—(N′,N′-dimethylaminoethane)carbamoyl)cholesterol (DC-Chol), N-(1,2-dimyristyloxypropa-3-yl)-N,N-dimethyl-N-hydroxyethylammonium bromide (DMRIE), Lipofectin (registered trademark), Lipofectamine (registered trademark), Transfectam (registered trademark), DODAP, DODMA, DMDMA, 1,2-dilinoleyloxy-N,N-dimethylaminopropane (DLinDMA), 1,2-dilinolenyloxy-N,N-dimethylaminopropane (DLenDMA), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 2,2-dilinoleyl-4-(2-dimethylaminoethyl)-[1,3]-dioxolane (DLin-KC2-DMA), (6Z,9Z,28Z,31Z)-heptatriaconta-6,9,28,31-tetraen-9-yl 4-(dimethylamino)butanoate (DLin-MC3-DMA), 1,2-dilinoleyloxy-3-trimethylaminopropane chloride salt (DLin-TMA-CI), 1,2-dilinoleoyl-3-trimethylaminopropane chloride salt (DLin-TAP-CI), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), 2,2-dilinoleyl-4-dimethylaminomethyl-[1,3]-dioxolane (DLin-K-DMA), 1-palmitoyl-2-oleoyl-sn-glycero-3-phosphocholine (POPC), and 1,2-distearoyl-sn-glycero-3-phosphocholine (DSPC). The aforementioned 2,2-dilinoleyl-4-(2-dimethylaminoethyl)-[1,3]-dioxolane (DLin-KC2-DMA), (6Z,9Z,28Z,31Z)-heptatriaconta-6,9,28,31-tetraen-9-yl 4-(dimethylamino)butanoate (DLin-MC3-DMA), and analogs of the preceding are described in Japanese Patent Application Laid-open Nos. 2013-245190, 2016-84297, and 2019-151589.

While not intended as a particular limitation, the following, for example, can be used as amphipathic substances, which are another example of self-assembling molecules: amphipathic polymer compounds, e.g., amphipathic block copolymers such as polystyrene-polyethylene oxide block copolymers, polyethylene oxide-polypropylene oxide block copolymers, polylactic acid-polyethylene glycol copolymers, polycaprolactone-polyethylene glycol copolymers, and so forth.

The substance to be encapsulated is not particularly limited, and can be exemplified by biopolymers such as nucleic acids, peptides, proteins, and glycans; by substances such as metal ions, small-molecule organic compounds, middle-molecule organic compounds, organometal complexes, and metal particles; and, broken down by application, by drugs, bioactive substances, cosmetic materials, and the like, i.e., anticancer agents, antioxidants, antibacterial agents, anti-inflammatory agents, vitamins, artificial blood (hemoglobin), vaccines, hair growth agents, moisturizers, colorants, whitening agents, pigments, and so forth. When these substances to be encapsulated are water soluble, they can be encapsulated in the aqueous phase of the particles that are formed. When they are poorly soluble in water, they can be encapsulated in the hydrophobic region of the self-assembled membrane that the self-assembling molecules form, or can be encapsulated within the particles as aggregates provided by binding and aggregation with the hydrophobic region of the self-assembling molecules. Water solubility or sparing solubility can be preliminarily imparted, or an aggregate can be made, by subjecting the substance to be encapsulated to a pretreatment step, and this pretreatment step can also be carried out in a flow path adapted for this treatment. When the pretreatment step is carried out in a flow path, the discharge port of the flow path that carries out this pretreatment step can be connected to the first or second introduction channel of the flow path structure body according to the present invention.

The water-miscible organic solvent used to prepare the particle solution by dissolution of the self-assembling molecules is not particularly limited, but the use is preferred, for example, of water-miscible organic solvents such as alcohols, ethers, esters, ketones, acetals, and so forth, and particularly alcohols such as ethanol, t-butanol, 1-propanol, 2-propanol, and 2-butoxyethanol and particularly alkanols having 1 to 6 carbons. In addition, the same water-miscible organic solvents can also be used as the water-miscible organic solvents used to prepare amphipathic substance solutions, with preferred examples being ethers such as tetrahydrofuran and chloroform.

Water—or an aqueous solution in which water is substantially the main compound, for example, physiological saline, phosphate buffer solutions, acetate buffer solutions, citrate buffer solutions, malate buffer solutions, and so forth—are used as appropriate as the dilution medium in correspondence to, for example, the application of the particles to be formed.

The self-assembling molecular particles encapsulating the substance to be encapsulated, and yielded by the method according to the present invention, can have a number-average particle diameter, for example, in the range of 20 to 200 nm. However, this should not be understood as a limitation to this range.

### EXAMPLES

The present invention is more particularly described hereinbelow based on examples. However, the examples are illustrations of the present invention, and the present invention should not be construed as being limited to or by the examples.

### Example 1

Production was carried out using a flow path structure body having the following structure and dimensions, and for which schematic diagrams of a portion of the structure are given in FIGS. 1-1(a) and (c).

### Structure and Dimensions

angle between the first introduction channel and the second introduction channel=90°
angle between the first introduction channel and the dilution flow path (line drawn to the upstream side of the X direction)=45°
angle between the second introduction channel and the dilution flow path (line drawn to the upstream side of the X direction)=45°
distance from the first introduction channel/second introduction channel merge point to the starting position for the dilution flow path=0.5 mm
starting position of dilution flow path=upstream side surface of the first Y structural element and the first Z structural element
The Y structural element is installed on the side of the first introduction channel.

flow path width y=200 μm,

height of Y structural elements (Y direction length)=150 μm,
width of Y structural elements (X direction length)=100 μm,
interval between adjacent Y structural elements=100 μm,
height of Z structural elements (Z direction length)=50 μm,
width of Z structural elements (X direction length)=100 μm,
interval between adjacent Z structural elements=300 μm,
number of Y structural elements=20
number of Z structural elements=10

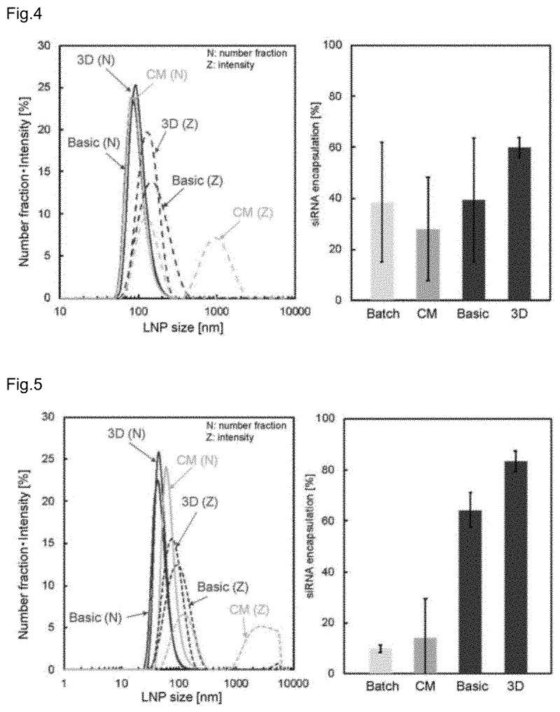

Aqueous solutions were prepared by diluting, using the buffer solutions indicated below from the second introduction channel, the anionic lipid starting material solution, neutral lipid starting material solution, or cationic lipid starting material solution, see below, from the first introduction channel of the above-described flow path structure body. The total flow rate was 500 μL/min, and the lipid starting material solution:buffer solution flow rate ratio was 1:2. After preparation, the alcohol in the aqueous solution was removed by performing dialysis using PBS as the external solution. The siRNA encapsulation ratio in the particles in the aqueous solution was measured using a RobiGreen assay (fluorescence measurement using an RNA quantitation reagent), and the results (3D) are reported in FIG. 3. Also reported in FIG. 3 are the results for the use of the chaotic mixer described in PTL 3 (CM) and the results for the use of the 2D flow path structure body described in PTL 1 (2D or Basic). The particle size distribution of the particles in the aqueous solution after preparation and posttreatment was measured using a Zetasizer Nano ZS (Malvern Panalytical Ltd.), and the results for the use of the anionic lipid solution are reported in FIG. 4 and the results for the use of the neutral lipid solution are reported in FIG. 5. FIGS. 4 and 5 report the particle size distribution on the left and the siRNA encapsulation ratio on the right. FIGS. 4 and 5 also report the results for Batch in addition to those for 3D, Basic, and CM. A summary of each experimental method is given in FIG. 2.

Starting Material Solutions

lipid solutions:

anionic: 10 mM DSPC/DOPS/Cholesterol (45/10/45 mol %) (solvent: EtOH (80% v/v)+MeOH (20% v/v))
neutral: 10 mM DSPC/Cholesterol (50/50 mol %) (solvent: EtOH)
cationic: YSK05/Cholesterol/DMG-PEG2K (50/50/1 mol %) (solvent: EtOH)

buffer solutions: 10 mM Tris-HCl (pH 8.0)+13.4 mM CaCl₂)+70 μg/mL siFVII (anionic neutral lipid)
25 mM acetate buffer solution (pH 4.0)+70 μg/mL siFVII

The results reported in FIG. 3 show that the 3D device according to the present invention can more efficiently encapsulate siRNA than other, conventional devices. The results reported in FIG. 4 for the use of an anionic lipid solution show that the particles produced by the 3D device exhibit a high size uniformity and can encapsulate siRNA with a good reproducibility. The results reported in FIG. 5 for the use of a neutral lipid solution show that the particles produced by the 3D device exhibit a high size uniformity and can encapsulate siRNA with a good reproducibility.

### Example 2

The same flow path structure body as in Example 1 was fabricated.

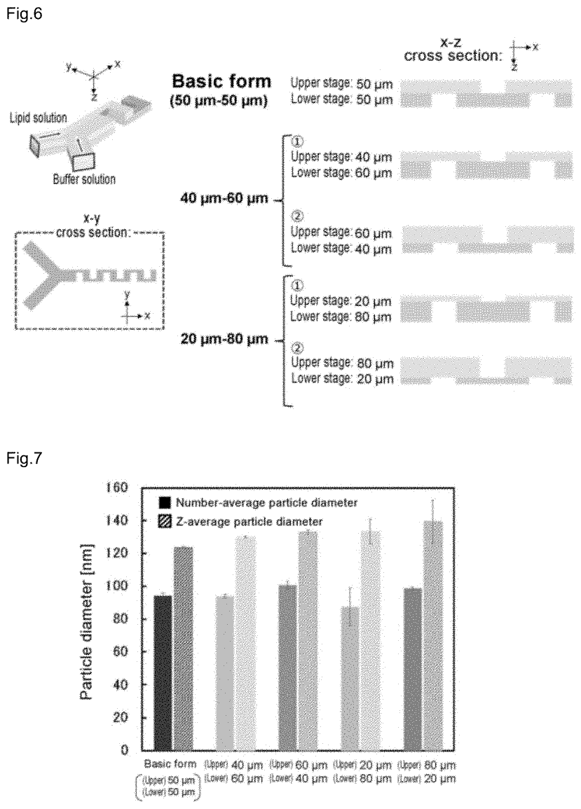

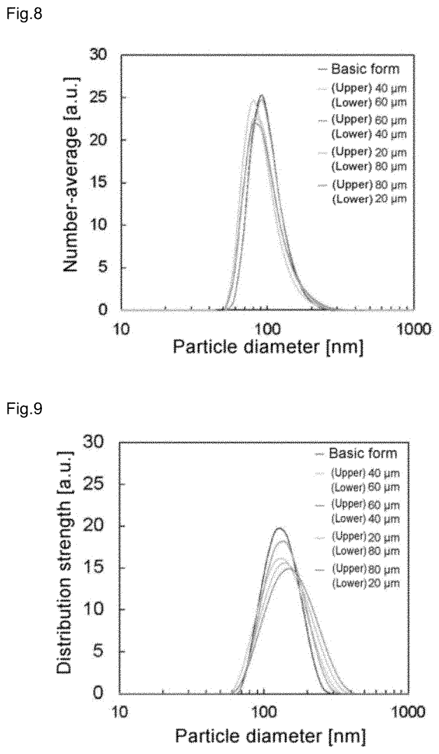

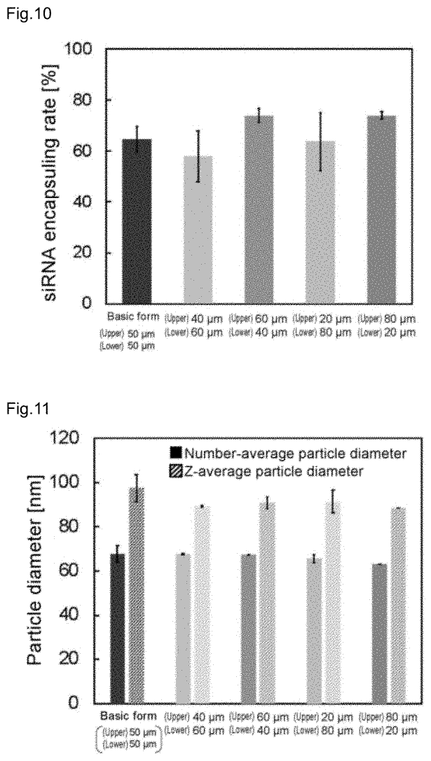

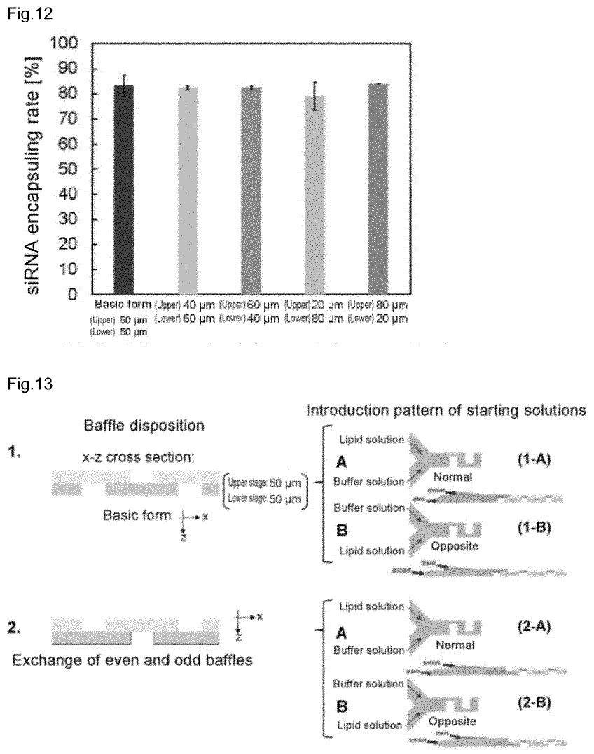

However, as shown in FIG. 6, the 50 μm height (Z direction length) of the Z structural elements (base shape=Example 1) was changed to 20 μm, 40 μm, 60 μm, or 80 μm. The same anionic lipid solution and neutral lipid solution as in Example 1 were used, and the same buffer solutions, total flow rate, and flow rate ratio as in Example 1 were used. After preparation, the siRNA encapsulation ratio in the particles in the aqueous solutions and the particle size distribution of the particles were measured as in Example 1. For the case of the anionic lipid solution, the results for the average particle diameter (number-average particle diameter and Z-average particle diameter) are reported in FIG. 7, the results for the number-average particle size distribution are reported in FIG. 8, the results for the Z-average particle size distribution are reported in FIG. 9, and the results for the siRNA encapsulation ratio are reported in FIG. 10. For the case of the neutral lipid solution, the results for the average particle diameter (number-average particle diameter and Z-average particle diameter) are reported in FIG. 11, and the results for the siRNA encapsulation ratio are reported in FIG. 12.

The results in FIGS. 7 to 9 (use of anionic lipid solution) show that for this condition the height (Z direction length) of the Z structural element of the flow path structure body does not substantially influence the particle diameter. According to the results in FIG. 10, there was a tendency for a higher siRNA encapsulation ratio to occur when the height (Z direction length) of the Z structural element was 20 μm and 40 μm (larger cross-sectional area for the flow path), which are smaller than (base shape=50 μm). The results in FIG. 11 (case of neutral lipid solution) show that for this condition the height (Z direction length) of the Z structural element of the flow path structure body does not substantially influence the particle diameter. The results in FIG. 12 show that for this condition the height (Z direction length) of the Z structural element of the flow path structure body has almost no influence on the siRNA encapsulation ratio.

### Example 3

The same flow path structure body as in Example 1 was fabricated.

However, as shown in FIG. 13, while the Y structural element is disposed in the base shape=Example 1 on the side of the first introduction channel (side of lipid solution inflow), a reverse disposition configuration was fabricated in which this was disposed on the side of the second introduction channel (side of buffer solution inflow).

In addition, experiments were carried out using the following variations.

1-A=base shape for flow path structure body, lipid solution inflow from first introduction channel
1-B=base shape for flow path structure body, lipid solution inflow from second introduction channel
2-A=reverse disposition configuration for flow path structure body, lipid solution inflow from first introduction channel
2-B=reverse disposition configuration for flow path structure body, lipid solution inflow from second introduction channel

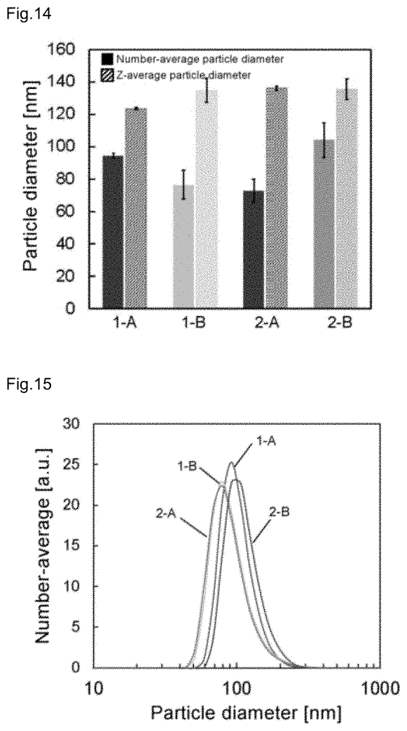

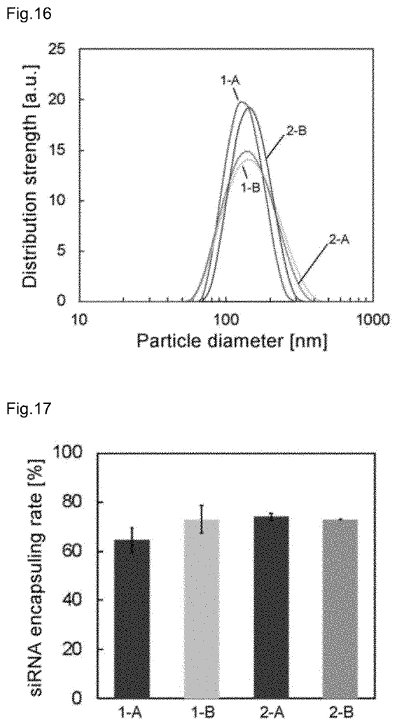

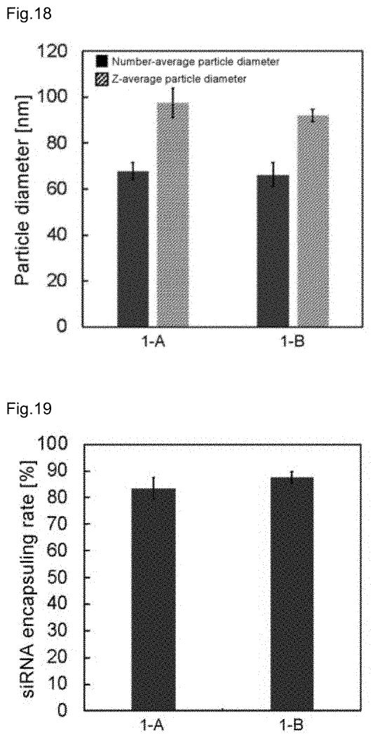

The same anionic lipid solution and neutral lipid solution as in Example 1 were used, and the same buffer solutions, total flow rate, and flow rate ratio as in Example 1 were used. After preparation, the siRNA encapsulation ratio in the particles in the aqueous solutions and the particle size distribution of the particles were measured as in Example 1. For the case of the anionic lipid solution, the results for the average particle diameter (number-average particle diameter and Z-average particle diameter) are reported in FIG. 14, the results for the number-average particle size distribution are reported in FIG. 15, the results for the Z-average particle size distribution are reported in FIG. 16, and the results for the siRNA encapsulation ratio are reported in FIG. 17. For the case of the neutral lipid solution, the results for the average particle diameter (number-average particle diameter and Z-average particle diameter) are reported in FIG. 18, and the results for the siRNA encapsulation ratio are reported in FIG. 19.

The results in FIGS. 14 to 16 (use of anionic lipid solution) show that for this condition the particles produced by the flow path structure body 1-A have a high particle diameter uniformity. According to the results in FIG. 17, the siRNA encapsulation ratios were approximately the same. The results in FIG. 18 (case of neutral lipid solution) show that for this condition there is not a large difference in the particle diameter between the particles produced using the flow path structure bodies 1-A and 1-B. The results in FIG. 19 show that for this condition there is almost no influence on the siRNA encapsulation ratio for the particles produced using the flow path structure bodies 1-A and 1-B.

### Example 4

The same flow path structure body as in Example 1 was fabricated.

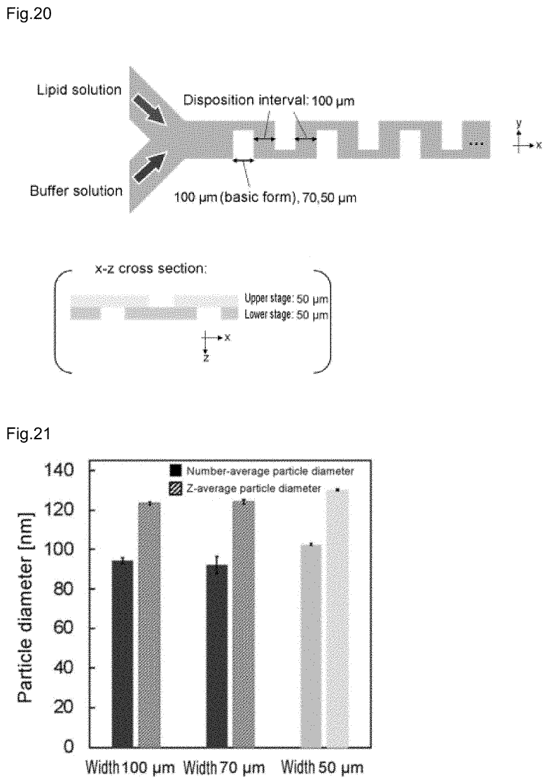

However, as shown in FIG. 20, while the width (X direction length)=100 μm for the Y structural element in the base shape=Example 1, flow path structure bodies were fabricated in which this was changed to 70 μm or 50 μm. Accompanying this change, the width (X direction length) of the Z structural elements was changed from 100 μm (base shape) to 70 μm or 50 μm, and the interval between adjacent Z structural elements was changed from 300 μm (base shape) to 240 μm or 200 μm. 100 μm (base shape) was retained for the interval between adjacent Y structural elements.

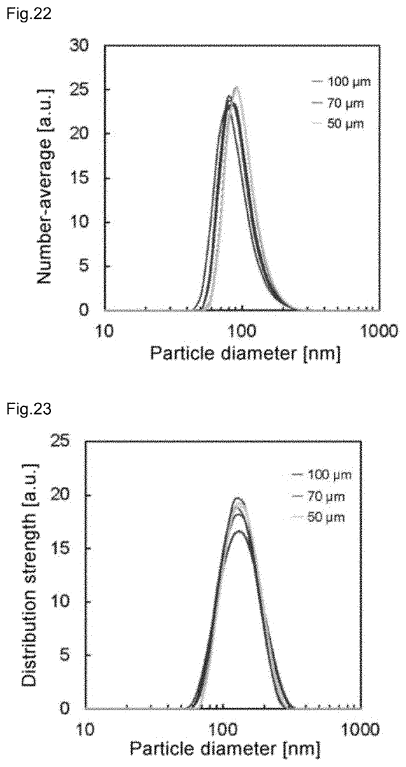

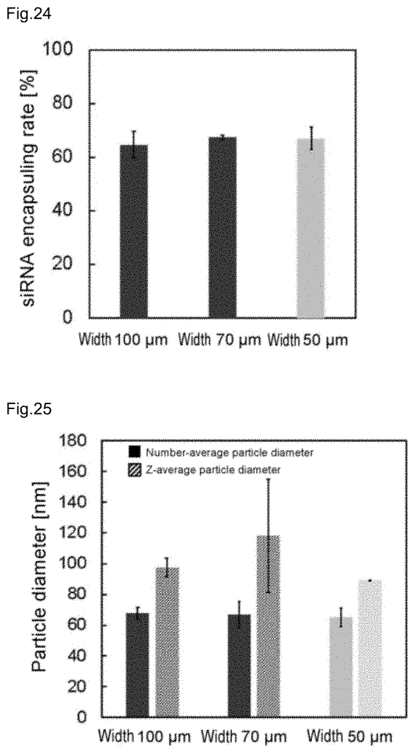

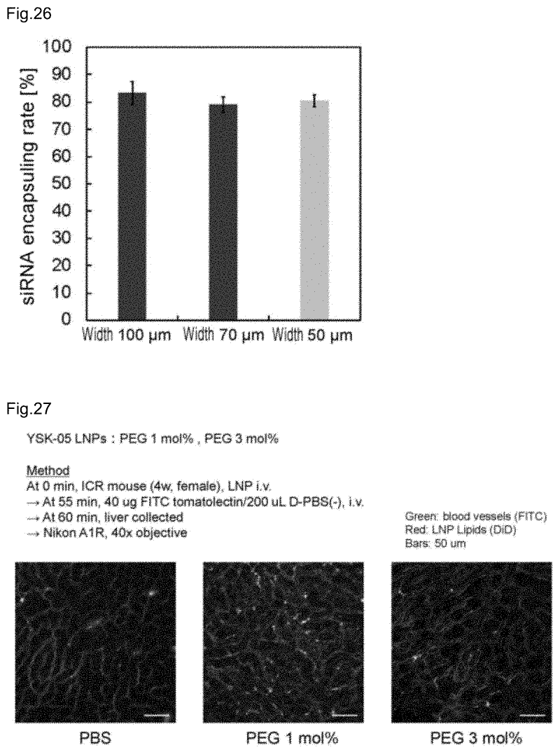

The same anionic lipid solution and neutral lipid solution as in Example 1 were used, and the same buffer solutions, total flow rate, and flow rate ratio as in Example 1 were used. After preparation, the siRNA encapsulation ratio in the particles in the aqueous solutions and the particle size distribution of the particles were measured as in Example 1. For the case of the anionic lipid solution, the results for the average particle diameter (number-average particle diameter and Z-average particle diameter) are reported in FIG. 21, the results for the number-average particle size distribution are reported in FIG. 22, the results for the Z-average particle size distribution are reported in FIG. 23, and the results for the siRNA encapsulation ratio are reported in FIG. 24. For the case of the neutral lipid solution, the results for the average particle diameter (number-average particle diameter and Z-average particle diameter) are reported in FIG. 25, and the results for the siRNA encapsulation ratio are reported in FIG. 26.

The results in FIGS. 21 to 23 (use of anionic lipid solution) show that for this condition differences in the width (X direction length) of the Y structural elements of the flow path structure body have no influence on the particle diameter of the particles. According to the results in FIG. 24, the siRNA encapsulation ratios were approximately the same. The results in FIG. 25 (case of neutral lipid solution) show that for this condition differences in the width (X direction length) of the Y structural elements of the flow path structure body have no influence on the particle diameter of the particles. The results in FIG. 26 show that for this condition differences in the width (X direction length) of the Y structural elements of the flow path structure body have almost no influence on the siRNA encapsulation ratio of the particles that have been produced.

### Example 5

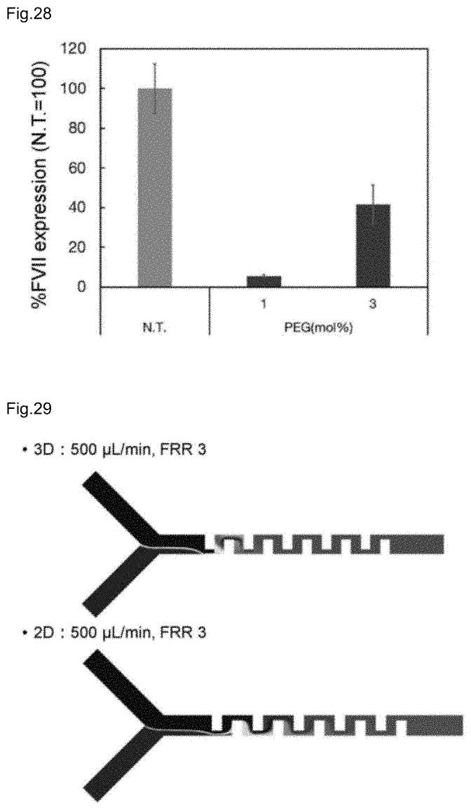

Lipid particle aqueous solutions were prepared using the 3D flow path structure body used in Example 1; the lipid particle aqueous solutions contained lipid nanoparticles constituted of a pH-responsive cationic lipid (YSK05), cholesterol, PEG lipid, and siRNA. The aqueous solutions were used to carry out an evaluation of the performance of the particles in the mouse, and the results are reported in FIGS. 27 and 28. It was confirmed that the target gene was successfully knocked down and that the delivery efficiency varied with the particle diameter.

### Reference Example 1 (Simulation)

In order to run a simulation of the state of dilution of the lipid solution in a 3D flow path structure body having the basic structure according to the present invention, ethanol, which was the water-miscible organic solvent of the lipid solution, and water as the dilution medium were flowed into the flow path structure body at a flow rate ratio of 1:3 and a total flow rate of 500 μl/minute or 50 μl/minute, and the flow was simulated using COMSOL Multiphysics multipurpose physics simulation software. The results are given in FIGS. 29 to 32 in comparison with a 2D flow path structure body.

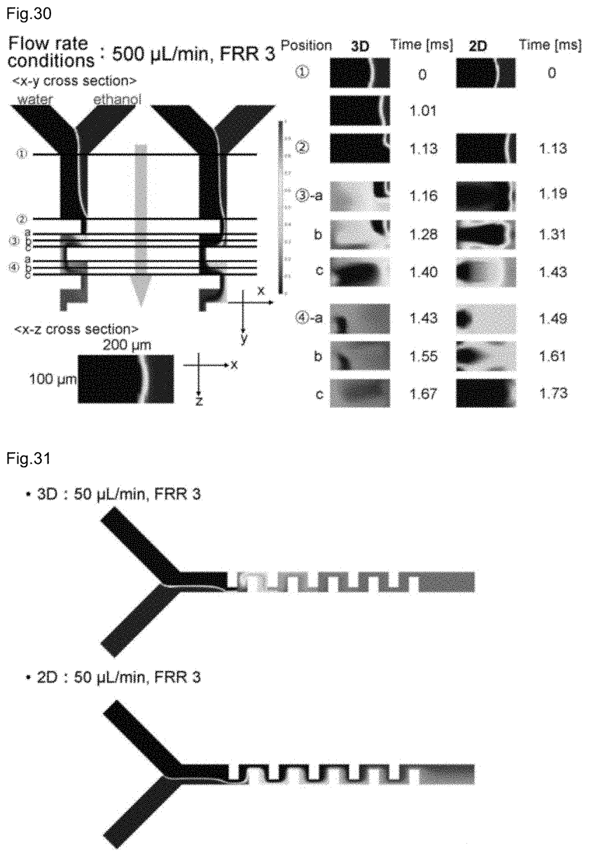

FIGS. 29 and 30 report the results for the total flow rate of 500 μl/minute, and show that, for the 3D flow path structure body, dilution is almost completed at between the 2nd and 3rd Y structural elements. For the 2D flow path structure body, on the other hand, the completion of dilution is between the 4th and 5th Y structural elements.

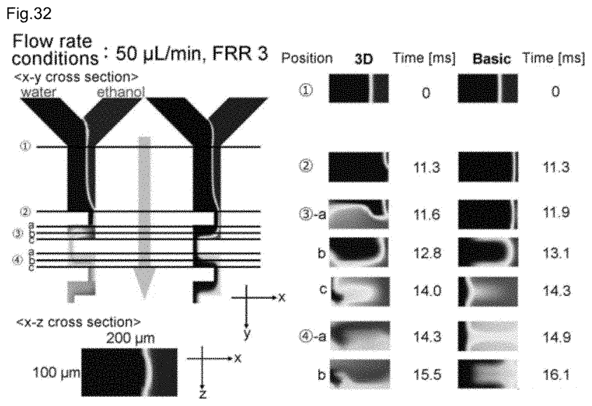

FIGS. 31 and 32 report the results for the total flow rate of 50 μl/minute, and show that, for the 3D flow path structure body, dilution is almost complete at between the 4th and 5th Y structural elements. For the 2D flow path structure body, on the other hand, the completion of dilution is not fulfilled at 10 Y structural elements.

### INDUSTRIAL APPLICABILITY

The present invention is useful in fields and sectors involved with or related to the technology of preparing particles that encapsulate, e.g., siRNA and so forth, in a self-assembling molecular particle, e.g., a lipid particle and so forth.

1.-21. (canceled)

22. A self-assembled molecular particle that encapsulates a substance to be encapsulated, wherein the self-assembling molecule is at least one selected from the group consisting of neutral lipid and anionic lipid, and wherein the substance to be encapsulated is a nucleic acid.

23. The self-assembled molecular particle according to claim 22, wherein encapsulation ratio of the nucleic acid in the particles is 60% or more.

24. The self-assembled molecular particle according to claim 22, wherein encapsulation ratio of the nucleic acid in the particles is 70% or more.

25. The self-assembled molecular particle according to claim 22, wherein encapsulation ratio of the nucleic acid in the particles is 80% or more.

26. The self-assembled molecular particle according to claim 22, wherein the nucleic acid is siRNA.

27. The self-assembled molecular particle according to claim 22, wherein the self-assembling molecular particle having the encapsulated substance to be encapsulated has a number-average particle diameter in the range of 20 to 200 nm.

28. The self-assembled molecular particle according to claim 22, wherein the self-assembling molecule is an anionic lipid.

29. The self-assembled molecular particle according to claim 22, wherein the self-assembling molecule does not contain a cationic lipid.

30. The self-assembled molecular particle according to claim 22, wherein the neutral lipid is selected from the group of phosphatidylcholines, phosphatidylethanolamines, sphingolipids, sterols, soy lecithin, hydrogenated soy lecithin and egg yolk lecithin.

31. The self-assembled molecular particle according to claim 22, wherein the anionic lipid is selected from the group of phosphatidylserines, phosphatidylglycerols, phosphatidic acids, phosphatidylinositols, glycolipids, gangliosides and long-chain alkyl phosphate salts.

32. A method for producing a self-assembling molecular particle, including supplying a self-assembling molecule-containing solution and a dilution medium to a flow path structure body, and forming a self-assembling molecular particle that encapsulates a substance to be encapsulated, wherein the self-assembling molecule is at least one selected from the group consisting of neutral lipid and anionic lipid, and wherein the substance to be encapsulated is a nucleic acid.

33. The production method according to claim 32, wherein encapsulation ratio of the nucleic acid in the particles is 60% or more.

34. The production method according to claim 32, wherein the flow path structure body has a base body and a flow path structure disposed in the interior thereof;
the flow path structure has, on its upstream side, at least two introduction channels that are independent from each other and are a first introduction channel that introduces a first liquid and a second introduction channel that introduces a second liquid, wherein these introduction channels merge at a merge site;
the flow path structure has at least one three-dimensionally bent dilution flow path toward the downstream side of the merge site; and
designating the X direction to be the axial direction of the dilution flow path upstream from the three-dimensionally bent dilution flow path, or the direction of the extension of this axial direction, designating the Y direction to be the width direction of the dilution flow path that perpendicularly intersects the X direction, and designating the Z direction to be the depth direction of the dilution flow path that perpendicularly intersects each of the X direction and Y direction, two or more Y structural elements that protrude in the Y direction and one or more Z structural element that protrudes in the Z direction are each independently present in at least a portion of the dilution flow path and at least two adjacent Y structural elements alternately protrude in the Y direction;
at least one of the self-assembling molecule-containing solution and the dilution medium contains a nucleic acid;
the self-assembling molecular particle is obtained by introducing the self-assembling molecule-containing solution from one of the first introduction channel and the second introduction channel of this flow path structure body, introducing the dilution medium from the other introduction channel, and diluting the self-assembling molecule-containing solution with the dilution medium in the dilution flow path.
